(19) United States
(12) Patent Application Publication
(10) Pub. No.: US 2014/0052018 A1
(43) Pub. Date: Feb. 20, 2014

(54) SPECIMEN REMOVAL BAG AND METHODS OF USING SAME

(71) Applicant: Intuitive Surgical Operations, Inc., Sunnyvale, CA (US)

(72) Inventor: Charles Patrick HAWKINS, Davidson, NC (US)

(73) Assignee: Intuitive Surgical Operations, Inc., Sunnyvale, CA (US)

(21) Appl. No.: 13/966,490

(22) Filed: Aug. 14, 2013

Related U.S. Application Data
(60) Provisional application No. 61/683,505, filed on Aug. 15, 2012.

(51) Int. Cl. A61B 17/221; A61B 1/303; A61B 17/42; A61B 1/313; A61B 10/04; A61B 17/32

(52) U.S. Cl. CPC A61B 17/221; A61B 10/04; A61B 17/320016; A61B 17/4241; A61B 1/3132; A61B 1/303
USPC 600/562; 606/114

(57) ABSTRACT

A specimen retrieval system for use with minimally invasive surgical procedures is disclosed. The system permits excised tissue having a dimension, a size, a shape, or a volume larger than a natural orifice or a surgical incision used in a minimally invasive procedure to access the surgical site, to be removed from the body through the natural orifice or surgical incision. The system comprises a specimen retrieval container and a compression structure. The compression structure is configured to apply a compressive force to the container to reduce or alter at least one of a dimension, a size, a shape, and a volume of the container and the tissue contained therein, such that the tissue can be removed through the natural orifice or surgical incision without need to surgically alter or enlarge the orifice or original incision.

This application claims the benefit of U.S. Provisional Application No. 61/683,505, filed Aug. 15, 2012, which is incorporated by reference herein in its entirety.

### TECHNICAL FIELD

The present teachings relate to minimally invasive removal of tissue, and more particularly to a system and method for removing excised tissue having a size larger than an access port used for the minimally invasive surgery, including a natural body passageway.

### INTRODUCTION

Minimally invasive surgical techniques are intended to reduce damage to surrounding tissue during diagnostic or surgical procedures, thereby reducing patient recovery time, discomfort, and deleterious side effects. As a result, the average length of a hospital stay for standard surgery may be shortened significantly through the use of minimally invasive surgical techniques. Also, patient recovery times, patient discomfort, surgical side effects, and time away from work may be reduced with minimally invasive surgery.

A common form of minimally invasive surgery is endoscopy, and a common form of endoscopy is laparoscopy, which is minimally invasive inspection and surgery inside the abdominal cavity. In endoscopic and laparoscopic surgeries, small openings in the body are used to gain access to a surgical site. These small openings in the body may be created, for example through use of a trocar, or natural body passages, such as the rectum or the vagina, may be used to provide access. In standard laparoscopic surgery, a patient's abdomen is insufflated with gas, and cannula sleeves are passed through small (approximately one-half inch or less) incisions to provide entry ports for laparoscopic instruments.

Laparoscopic surgical instruments generally include an endoscope (e.g., laparoscope) for viewing the surgical field and tools for working at the surgical site. The working tools are typically similar to those used in conventional (open) surgery, except that the functional component at the working end of each tool is separated from its handle by an extension tube that forms the main instrument shaft. The functional component can include, for example, a clamp, grasper, scissor, stapler, cautery tool, linear cutter, or needle holder.

To perform surgical procedures, the surgeon passes working tools through the body wall, either directly via the entry port or indirectly through a tubular access such as a cannula sleeve to an internal surgical site and manipulates them from outside the abdomen. The surgeon views the procedure by means of a monitor that displays an image of the surgical site taken from the endoscope. Similar endoscopic techniques are employed in, for example, arthroscopy, retroperitoneoscopy, pelviscopy, nephroscopy, cystoscopy, cisternoscopy, sinoscopy, hysteroscopy, urethroscopy, and the like.

Minimally invasive surgical procedures also may be performed using telesurgical robotic systems. Such systems increase a surgeon's dexterity when working on an internal surgical site, allow a surgeon to operate on a patient from a remote location (outside the sterile field). In a telesurgery system, the surgeon is often provided with an image of the surgical site at a control console. While viewing an image of the surgical site on a suitable viewer or display, the surgeon performs the surgical procedures on the patient by manipulating master input or control devices of the control console. Each of the master input devices controls the motion of a servo-mechanically actuated/articulated slave surgical instrument. During the surgical procedure, the telesurgical system can provide mechanical actuation and control of a variety of surgical instruments or tools having functional components (often referred to as end effectors in the field of robotic surgery) that perform various functions for the surgeon similar to the functions that manually actuated working tools provide, for example, holding or driving a needle, grasping a blood vessel, dissecting tissue, or the like, in response to manipulation of the master input devices.

Minimally invasive endoscopic, laparoscopic, and telesurgical procedures can be used to partially or totally remove body tissue (e.g., organs or other body components) from the interior of the body, e.g., nephrectomy, cholecystectomy, hysterectomy, spleenectomy, liver resection, lung resection, removal of cysts and/or tumors, and other such procedures. During such procedures, after excising the tissue, it is desirable to remove the tissue through an existing access port to the surgical site, whether the access port is a natural body orifice or is surgically-created. Excised tissues and/or organs may vary in composition and hardness and may include, for example, soft tissue, fluid-filled tissue (e.g., cysts, spleen), firm tissue (e.g., muscle), gallstones, fibroids, tumors, and combinations of these tissues.

Often, the excised tissue is placed in bag, such as a specimen retrieval bag, prior to removal from the surgical site. This is particularly useful in instances when the tissue or organ being removed is diseased or cancerous, and is therefore potentially dangerous to surrounding tissues. For example, if not contained, cancerous tissue can shed cancerous cells during removal, potentially permitting the recurrence of the cancer. The excised tissue is placed in the specimen retrieval bag, and then the bag is closed and withdrawn from the body through the access port. In certain instances, however, the excised tissue is too large to be withdrawn from the body through the access ports to the surgical site. In some cases, the surgeon may enlarge the access port to allow the tissue to be removed. In other cases, the tissue can be cut up (morcellated) to reduce its size, and permit removal through the access port. Morcellation instruments can be used to cut up the tissue within a specimen bag.

Morcellation, however, may be undesirable if the tissue to be removed is diseased or cancerous, due to the fact that cutting the tissue may release fluids from the excised tissue and/or pieces of tissue, into the body cavity from which the tissue is being removed. Further, manipulating both the specimen the bag and morcellation tools can be difficult, the bag may be punctured, or tissue and/or fluids can spill if the bag slips during morcellation or removal.

Additionally, certain tissues, such as a uterus, may be too large to fit within a single standard specimen bag, even when morcellated, or they may not fit through the minimal access ports for removal from the body even when morcellated. In such cases, it may be necessary to create an additional surgical access to the surgical site in order to remove the tissue. Morcellating tissue to reduce its size for removal from the body, as well as creating additional surgical access to remove the tissue, increases the time necessary for the surgical procedure and, thus, increases the risks inherent to any surgery, such as infection. And, creating a relatively larger port just to remove tissue is contrary to the purpose of minimally invasive surgery.

Thus, a specimen retrieval bag that facilitates removal of large tissue masses, or tissues partially comprising hard or functionally incompressible elements, through an existing access port in a minimally invasive surgical procedure is desirable.

### SUMMARY

The present teachings provide a specimen retrieval system for use with minimally invasive surgical procedures. The system permits removal of excised tissue having a dimension, a size, a shape, or a volume larger than an access port used for the minimally invasive surgery, including a natural body passageway, through the access port.

According to one aspect of the present disclosure, the system comprises a specimen retrieval container having a first open end, a second end, and a body extending between the first end and the second end to define a length of the container. The system further comprises a compression structure extending along a substantial portion of the length of the container, the compression structure movable between a first configuration and a second configuration, wherein, when in the second configuration, the compression structure applies a compressive force along a length of the container to reduce or alter at least one of a dimension, a size, a shape, and a volume of the container and thereby compress a tissue contained therein.

According to another aspect of the present disclosure, a method of removing excised tissue from an endoscopic surgical site is provided. The method comprises inserting a specimen retrieval container into a patient, and positioning the specimen retrieval container relative to excised tissue at a surgical site within the patient. The method further comprises substantially encompassing the excised tissue with the specimen retrieval container, and applying a force to the specimen retrieval container to reduce or alter at least one of a dimension, a size, a shape, and a volume of the specimen retrieval container and thereby compress the excised tissue contained therein.

According to yet another aspect of the present disclosure, a system according to the present teachings includes a specimen retrieval bag having a first open end, a second closed end, and a body extending between the first end and the second end of the bag, the body of the bag having a delivery configuration, a specimen receiving configuration, and a specimen removal configuration. The system further comprises a compression structure for reducing or altering at least one of a dimension, a size, a shape, and a volume of the bag to change the configuration of the bag from the specimen receiving configuration to the specimen removal configuration.

Additional objects and advantages of the present teachings will be set forth in part in the description which follows, and in part will be obvious from the description, or may be learned by practice of the teachings. The objects and advantages of the present teachings will be realized and attained by means of the elements and combinations particularly pointed out in the appended claims.

It is to be understood that both the foregoing general description and the following detailed description are exemplary and explanatory only and are not restrictive of the present teachings, as claimed.

The accompanying drawings, which are incorporated in and constitute a part of this specification, illustrate exemplary embodiments of the present teachings and, together with the description, serve to explain the principles of the present teachings.

### BRIEF DESCRIPTION OF THE DRAWINGS

FIGS. 1A and 1B are perspective views of an exemplary embodiment of a specimen retrieval system shown in a specimen receiving configuration and a specimen removal configuration, respectively, utilizing a single laced-type closure in accordance with the present teachings.

FIGS. 2A and 2B are perspective views of an alternative exemplary embodiment of a specimen retrieval system shown in a specimen receiving configuration and a specimen removal configuration, respectively, utilizing a plurality of laced-type closures in accordance with the present teachings.

FIGS. 3A and 3B are perspective views of another exemplary embodiment of a specimen retrieval system shown in a specimen receiving configuration and a specimen removal configuration, respectively, utilizing a plurality of shape memory material closure elements in accordance with the present teachings shows in accordance with the present teachings.

FIGS. 4A and 4B are perspective views of alternative exemplary embodiments of a specimen retrieval system using a single shape memory closure element in accordance with the present teachings. FIG. 4A shows a specimen receiving container in a specimen receiving configuration and FIG. 4B shows an alternative embodiment of a specimen receiving container in a specimen removal configuration.

FIGS. 5A and 5B are perspective views of an exemplary embodiment of a specimen retrieval bag shown in a specimen receiving configuration and a specimen removal configuration, respectively, in accordance with the present teachings.

FIG. 6A is a perspective view of the specimen retrieval bag of FIG. 5A in a specimen receiving configuration; and FIG. 6B is a perspective view of the specimen retrieval bag of FIG. 6A in a delivery configuration, in accordance with the present teachings.

FIG. 7 is a cross-sectional illustration of a normal size uterus.

FIG. 8 is a cross-sectional illustration of a normal size uterus, uterine tubes, and ovaries.

FIGS. 9-11 illustrate the process of positioning the specimen retrieval container, in the configuration shown in FIG. 1A, from a position within the body accessed by a minimally invasive port, over tissue to be removed from the body, and the subsequent application of compression to the tissue contained in the retrieval container as the retrieval container configuration moves from that shown in FIG. 1A to that shown in FIG. 1B.

FIG. 12 illustrates an alternative approach to inserting the specimen retrieval container, shown in FIG. 1A, into the body of the patient, over the uterine retractor.

FIG. 13 shows the anatomy involved in treating pancreatic cancer through surgery.

FIG. 14A illustrates the anatomy removed during one type of whipple procedure.

FIG. 14B illustrates an alternative tissue block excised during a whipple procedure.

FIGS. 15A and 15B illustrate a process of positioning a specimen retrieval container, in accordance with the present teachings, from a position within the body accessed by a minimally invasive port, over tissue to be removed from the body as identified in FIG. 14A, and the subsequent application of compression to the tissue contained in the retrieval container as the retrieval container and its contents are shaped to be removed from the body through the minimally invasive port.

### DETAILED DESCRIPTION

Reference will now be made in detail to exemplary embodiments of the present teachings, examples of which are illustrated in the accompanying drawings. Wherever possible, the same reference numbers will be used throughout the drawings to refer to the same or like parts.

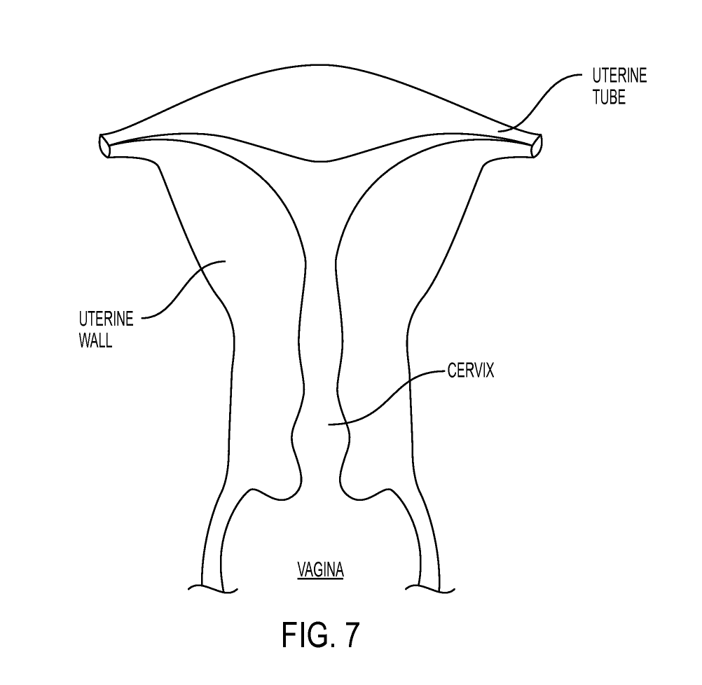

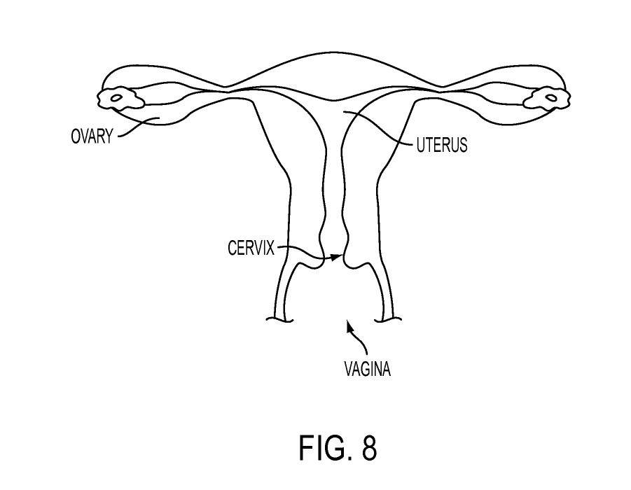

As discussed above, minimally invasive surgeries reduce the need for large incisions, thereby promoting better patient outcomes, including shorter hospital stays and faster healing. Many types of major surgeries, previously performed as open surgical procedures, can now be done using minimally invasive techniques. One such type of surgery is a hysterectomy. During a hysterectomy, the uterus is removed. A hysterectomy may be a full hysterectomy, in which the entire uterine structure is removed (body, fundus, and cervix), or a partial hysterectomy, where only the uterine body is removed. See FIGS. 7 and 8. In some cases, the ovaries and fallopian tubes may be removed at the same time, for example to reduce the risks of certain types of cancer. Hysterectomies may be performed by laparotomy (abdominal incision), by vaginal hysterectomy (performed through the vaginal canal), and by laparoscopic vaginal hysterectomy or robotic hysterectomy (both performed using small incisions (ports) in the abdomen and the vaginal canal). During vaginal hysterectomy, laparoscopic vaginal hysterectomy, and robotic hysterectomy, the uterus is typically and most desirably removed through the vagina. However, in many cases, the size of the uterus is too large to be removed vaginally, and it is necessary to make an abdominal incision to remove the uterus.

In estimating the size of a uterus to determine the potential for a minimally invasive hysterectomy procedure, surgeons measure the size of the uterus (through palpation) and equate it to the number of weeks of gestation the size of the uterus represents, although it is important to note that the relationship between uterine weight and gestational size may vary based on the pathology of the enlarged uterus. The size and weight of the uterus is often dependent upon the type of condition(s)/diseases for which the uterus is being removed. For example, endometriosis, fibroids, and tumors can increase both the size and weight of the uterus. As an example, a uterus that weighs up to 250 grams would be considered a “normal” sized uterus and estimated at a gestation of 0-4 weeks. A “large” uterus is a uterus that weighs more than 250 grams and may be estimated at a size of 6-8 weeks gestation. Laparoscopic procedures are increasingly complex as uterine weight increases and procedures to remove uteruses greater than about 400 grams (8-10 weeks gestation) are often converted to an open procedure. Robotic laparoscopy provides more instrument control and better visualization, which can allow surgeons to offer minimally invasive procedures to women with much larger uteruses. Published reports show average conversion to open procedures during robotic laparoscopy when uterine weight is around 1300 grams (20-24 weeks gestation).

Limitations for removing the uterus vaginally during a laparoscopic or robotic procedure are due in large part to the size of the vaginal canal. A normal size uterus is about the size of a golf ball, and can be removed vaginally. However, as the size of the uterus increases, the possibility of removing the uterus through the vaginal canal decreases. Additionally, as the size of the uterus increases, the shape of the uterine anatomy becomes more cumbersome and difficult to manipulate, creating additional difficulties when trying to remove the uterus vaginally. The present disclosure provides a system and method for containing a uterus, compressing the uterus to reduce its size and/or change its shape, and removing it vaginally during a laparoscopic or computer-assisted procedure.

In accordance with an alternative procedure, a system as disclosed herein also may be used in a computer-assisted whipple procedure performed on pancreatic cancer patients. For example, a system in accordance with the present teachings may be used in a laparoscopic pancreatic duodenectomy in which the gall bladder, a portion of the pancreas (containing a tumor), a portion of the small intestine (duodenum), the bile duct, and in some cases, a portion of the stomach, are removed. In some instances, these tissues may be removed together in a block. The removal of such a large block of tissue, some of which may contain tumor or other incompressible tissue, may be facilitated by use of a specimen retrieval container and method in accordance with the present teachings, wherein the system and method can contain the block of excised tissue, compress the excised tissue to reduce its size and/or change its shape, and remove the tissue contained in the specimen retrieval container through an existing minimally invasive surgical access port during a laparoscopic or computer-assisted procedure.

Additionally or alternatively, the system and method disclosed herein may be used in additional surgical procedures in which a block of tissue is excised during a minimally invasive procedure. As noted previously, in cases in which cancer or disease is involved, it may be desirable to fully contain and remove the excised block of diseased or cancerous tissue as a single unit, or within a single container, to minimize the chance that diseased or cancerous cells are shed during the removal process. In addition to hysterectomy and whipple procedures, discussed above, the system and method discussed herein may have a particular application in thoracic and/or colorectal procedures, in which a relatively large amount of tissue may be excised and removed. For example, in thoracic surgeries in which a lobe of a lung (possibly containing cancerous tissue or tumor(s)) is removed, the block of tissue may be as large as 3″×3″×5″-4″×4″×8″. Reduction and/or alteration of a dimension, a size, a shape, and/or a volume of the excised lung tissue via a system according to the present teachings may permit removal of the excised tissue through a surgical access port. As an additional example, in certain colorectal procedures, a large portion of the colon may be excised. The excised colon may measure 4-10 inches in length and have a diameter of 2-3 inches. Reduction and/or alteration of a dimension, a size, a shape, and/or a volume of the excised colon via a system according to the present teachings may permit removal of the excised tissue through a surgical access port. In each of the above examples, a specimen retrieval system according the present teachings can be used to contain the block of excised tissue, compress the excised tissue to reduce its size and/or change its shape, and remove the tissue contained in the specimen retrieval container through an existing minimally invasive surgical access port during a laparoscopic or computer-assisted procedure.

Although the system and method disclosed herein are discussed in relation to a hysterectomy procedure and in relation a whipple procedure, the present disclosure is not so limited. A system and method, according to the present teachings, can be used with any laparoscopic or computer-assisted surgery in which it is desirable to reduce a size and/or change a shape of excised tissue prior to removing it through an existing access port or natural body passage. For example, a system as disclosed herein may be used in conjunction with the removal of a gall bladder (containing incompressible gall stones), a portion of resected lung (in some cases containing tumor), a colon extraction, a spleen, an appendix, etc.

In addition, although the system and method disclosed herein are discussed in relation to surgical procedures, the present disclosure is not so limited. A specimen retrieval system and method, in accordance with the present teachings, can be used in non-surgical procedures to access a material within an area having limited access, such as through a natural opening or where an opening must be created to provide access to the area. Such natural and man-made openings may be found, for example, in caves or rock formations, in pipes and duct work, and/or in well bore (e.g., in the gas/oil industry). Using the specimen retrieval system, a desired material can be collected and positioned within a specimen retrieval container in accordance with the present teachings, and the specimen retrieval container can be manipulated to reduce a size and/or change a shape of the container and the material contained therein so as to permit removal through the natural opening or prior existing access that was created. Additionally or alternatively, in such a non-surgical embodiment, it is possible that a specimen retrieval container in accordance with the present teachings also could be used to deliver a material into an area having such limited access.

In accordance with the present teachings, a specimen retrieval system for use with minimally invasive surgical procedures, including for example, laparoscopic, thoracoscopic, and teleoperated computer-assisted (also referred to as robotic) procedures, is provided. The specimen retrieval system as disclosed herein permits the removal of a mass of excised tissue, including organs, having a size larger than the size of an existing minimally invasive surgical access port, including access ports formed by natural body orifices such as the mouth, the nostrils, the anus, the urethra, and the vagina, without creating a larger abdominal incision and without requiring morcellation of the tissue prior to removal.

As disclosed herein and shown in exemplary embodiments, a specimen retrieval system includes a specimen retrieval container having a first size and a first volume. The system also includes a structure for applying a compressive force to at least a portion of the container to change or reduce at least one of a dimension, a size, a shape, and a volume of the specimen retrieval container. The structure applies a compressive force to the container and its contents to change and/or reduce a dimension, the size, the shape, and/or volume of the container and, thus, changes and/or reduces a dimension, the size, the shape, and/or volume of the excised tissue contained therein. The container, when in a non-compressed configuration, is shaped and sized to receive excised tissue having a dimension or a size larger than an opening of an existing minimally invasive surgical access port or natural body orifice. When in the compressed configuration, at least one of a dimension, a size, a shape, and/or a volume of the container and the excised tissue contained therein is changed or reduced. For example, in the case of a laparoscopic or computer-assisted hysterectomy, the specimen retrieval system can reduce the size of a large excised uterus to a size that will pass through the vaginal canal. Although the size of the vaginal opening varies from person to person, and based on infants birthed, as a general observation materials such as excised tissue need to have a diameter of less than about three inches to pass through the vaginal canal, and generally speaking, a diameter of two inches should allow passage of most items. Thus, a specimen retrieval container according to the present disclosure may have a tissue-receiving, non-compressed configuration that has a size, a shape, a volume and/or a dimension larger than the vaginal canal, such that if the bag is full or substantially full of excised tissue, the bag and excised tissue cannot pass through the vaginal canal. The specimen retrieval container according the present disclosure may further have a compressed configuration that has a size, a shape, a volume and/or a dimension smaller than that of the tissue-receiving, non-compressed configuration to allow passage of the container and any tissue contained therein through the vaginal canal. For example, the structure for applying compression to the specimen retrieval container may be configured to apply compression to the container in a manner that will reduce a diameter of the specimen retrieval container, thus reshaping any tissue inside the container, to permit passage of the container and tissue contained therein through the vaginal canal.

Although the system and method disclosed herein are discussed in relation to removal of excised tissue through a vaginal canal and the reshaping of the excised tissue, via a reduction of a dimension of the tissue to permit passage through the vaginal canal, the present disclosure is not so limited. A system and method, according to the present teachings, can be used with any type of excised tissue to adjust any dimension, shape, size, and/or volume of the excised tissue in a manner that will permit the excised tissue to take on a dimension, shape, size, and/or volume that will enable exiting the body via an existing access port or natural body passage such as via a thoracoscopic port or the rectum, respectively. For example, the system and method might be used to remove sigmoid colon and/or rectum thru the vagina or the rectum. Additionally or alternatively, as discussed above, the system and method of the present disclosure may be used to remove a block of tissue comprising the gall bladder, a portion of the pancreas (containing a tumor), a portion of the small intestine (duodenum), the bile duct, and in some cases, a portion of the stomach, the block of tissue being excised during a laparoscopic pancreatic duodenectomy (in computer-assisted cases, this surgery is sometimes referred to as a “robotic whipple procedure”).

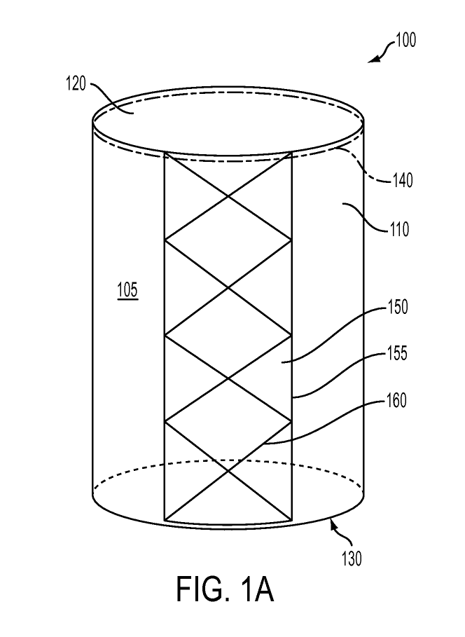

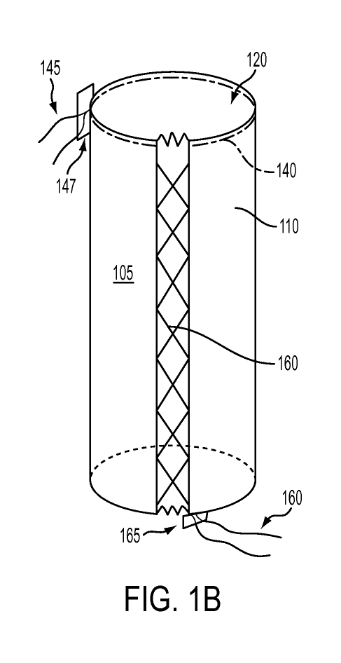

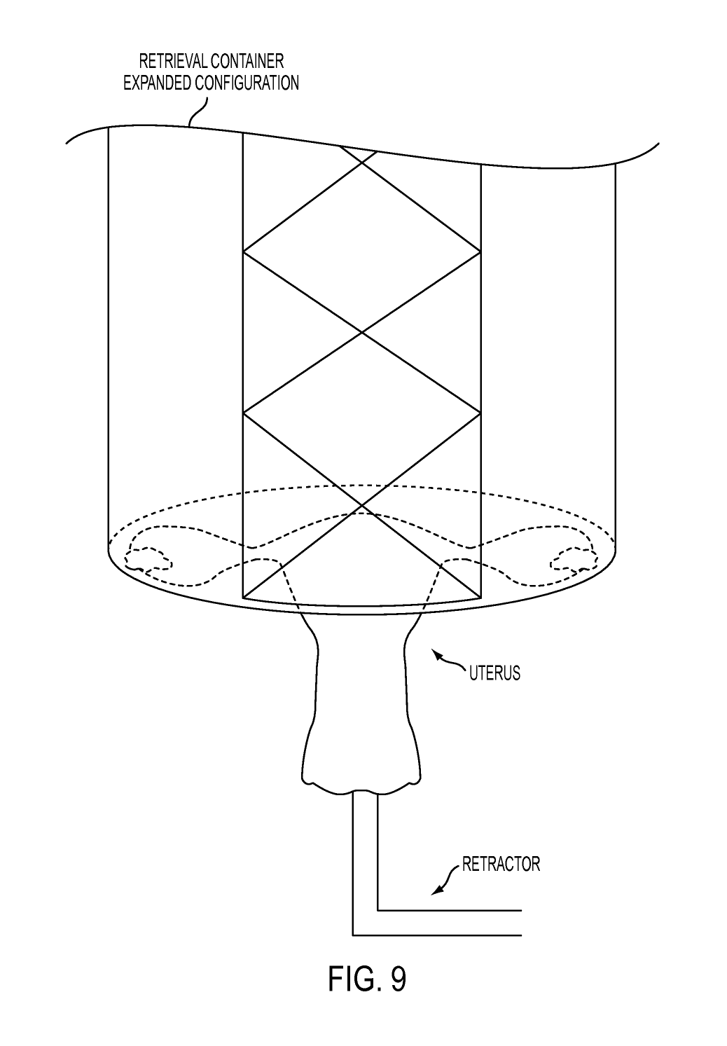

According to one aspect of the present disclosure, a specimen retrieval system 100 includes a container 110 for receiving excised tissue (see FIGS. 9-11). As illustrated in FIG. 1A, container 110 includes a first end 120, a second end 130, and a body 105 extending between the first and second ends. The body 105 may be a wall, as illustrated in FIG. 1A. For example, the body 105 may be cylindrical in shape, as illustrated in FIG. 1A, or it may taper, such that it is larger at one end than another. As will be understood by those of ordinary skill in the art, the container body 105 may be of any shape suitable to hold excised tissue. As shown in FIG. 1A, first end 120 of container 110 may be an open end for receiving an excised tissue specimen. Second end 130 of container 110 may include an opening, such that the container 110 forms a sleeve structure, or it may be closed, such that container 110 forms a type of bag structure. Open end 120 of container 110 may include a closure system 140 for closing open end 120 after a specimen is placed within the container. The closure system 140 may include any conventional structure for closing an open container. As illustrated in the exemplary embodiment of FIGS. 1A and 1B, closure system 140 may include a drawstring type closure element 145. Additionally, closure system 140 may include a clip or fastener type element 147 to maintain the closure element 145 in a closed configuration. As illustrated in FIG. 1B, the closure system 145 may reduce a size of the opening of open end 120. Additionally or alternatively, closure system 145 may completely close the opening of open end 120. End 130, when provided in an open configuration forming a sleeve-type container, also may include a closure system 140 having a closure element 145 and fastener type element 147 for closing end 130.

In accordance with the present teachings, retrieval container 110, such as a bag or a sleeve, may be made from any suitable material, such as a polymeric or an elastomeric material, including nylon, plastic, polyurethane, polyethylene, polyester, polypropylene, silicone, PBT, PET, and PTFE, and various combinations thereof. The container may be formed of one or more layers, and it may include reinforcing elements, such as fibrous materials, wires, meshes, or metals, spaced throughout the container.

According to another aspect of the present disclosure, system 100 may include a compression structure for applying compression to the body of the retrieval container to reduce a dimension, size, or volume of the container, such as a diameter of the container and, consequently, change a dimension, shape, size, or volume of a tissue specimen contained. As illustrated in the exemplary embodiment of FIGS. 1A and 1B, a compression structure may include a lacing construction 150, such as might be found in certain corsets or as may be used to pull together flaps on a sneaker. Lacing construction 150 extends substantially along a length of the container 110, and includes a drawstring 160 that passes through a force distributing element or compression element 155. Element 155 may include a pair of compression elements 155, such as flaps attached to exterior portions of body 105 of container 110, or element 155 may be a single element, such as a cinch having a length shorter than a circumference of the container 110, that extends substantially around container 110 and includes first and second edges for receiving the drawstring. Element 155 may include eyelets, holes, or other structure (not shown) for receiving the drawstring 160 in a threaded or laced manner. When a pulling force is applied the drawstring 160, the drawstring 160 pulls on the edges of the compression element 155, moving the edges of compression element 155 (or the pair of compression elements 155) toward one another and collapsing a portion of the container between the edges. Compression element(s) 155 distributes the force substantially uniformly along the length of the compression element 155 and the container 110, and also around a circumference of container 110. The distributed force acts to compress the container 110 and the specimen contained therein, reducing a dimension (e.g., a diameter), size (e.g., a cross-sectional area), shape, or volume of the container 110 and correspondingly reducing or changing a dimension, size, shape, or volume of the specimen within the container 110.

Although a drawstring 160 is disclosed as the mechanism for pulling compression element(s) 155 toward one another to apply a compressive force along the length of the container 110, it should be understood that other types of fastener mechanisms may be used instead. For example, the drawstring might be replaced by a zipper structure, a series of toggle type fasteners, or a series of buckle type fastening elements. Additionally or alternatively, the drawstring 160 may comprise any suitable biocompatible material including, for example, a string, a suture, a cord, etc.

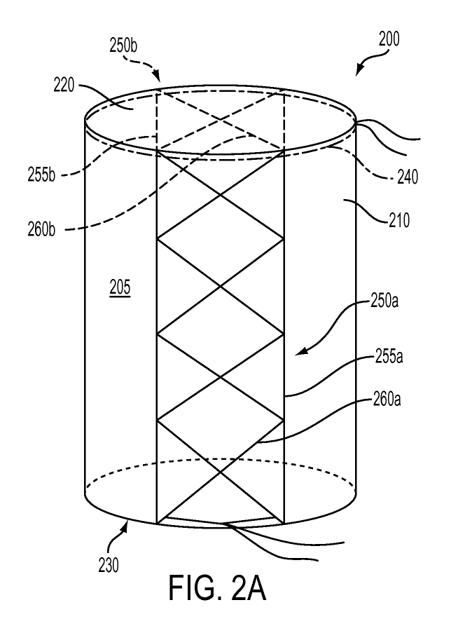

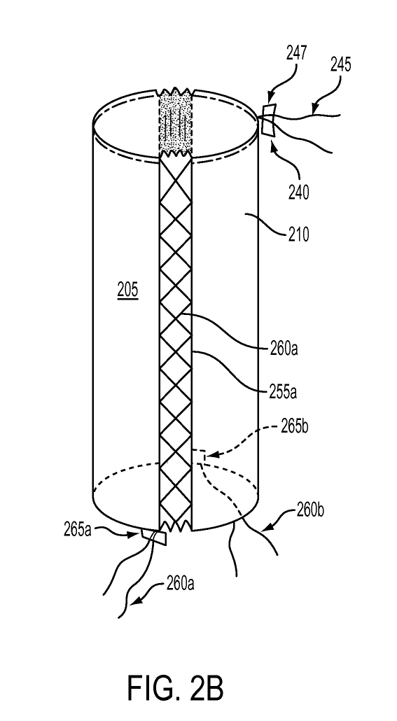

In order to maintain the compressive force on the container 110 and the specimen contained therein, a clip, slide, or fastener type element 165 may be used to maintain the position of the drawstring 160 once it is drawn closed to pull the compressive element(s) 155 together to create the compressive force on the container 110. Maintaining the drawn or closed position of the drawstring will hold the compressive element(s) 155 in place, thus maintaining the force on the container 110 and the tissue contained therein. Additionally or alternatively, as illustrated in FIGS. 2A and 2B, a container 210 may include first and second lacing structures 250a, 250b, located on opposite sides of the container, to apply compressive forces to the container and the tissue contained therein. It should be understood that more than two lacing structures may be used as needed to provide the necessary compressive force on the container. As discussed above, container 210 may include an open end 220 and a closed end 230, or two open ends 220 and 230. For each open end, container 210 may include a closure system 240 for closing open end 220, 230 after a specimen is placed within the container. The closure system 240 may include any conventional structure for closing an open container, and as illustrated in the exemplary embodiment of FIGS. 2A and 2B, closure system 240 may include a drawstring type closure element 245. Additionally, closure system 240 may include a clip or fastener type element 247 to maintain the closure element 245 in a closed configuration. As illustrated in FIG. 2B, the closure system 245 may reduce a size of the opening of open end 220, 230. Additionally or alternatively, closure system 245 may completely close the opening of open end 220, 230.

In an alternative exemplary embodiment, a compression structure 350 may comprise a plurality of shape memory alloy compression elements 350a, 350b, 350c, movable between a first non-engaged configuration, in which a specimen receiving container 310 is open to receive a tissue specimen, and a second engaged configuration, in which the compressive elements 350a, 350b, 350c are engaged to apply a compressive force to container 310, resulting in a reduced or altered dimension (e.g., diameter), size (e.g., cross-sectional area), shape or volume of the container 310, the shape alloy compression elements 350a, 350b, 350c applying compression along a substantial length of the container 310, and via the container, compressing any tissue contained therein.

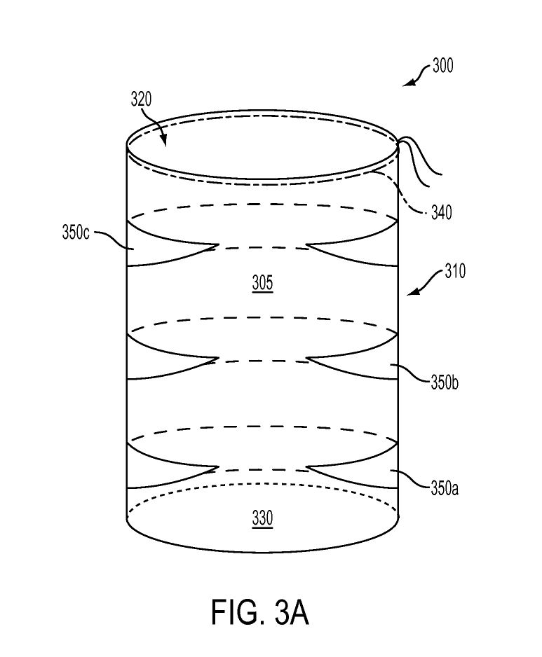

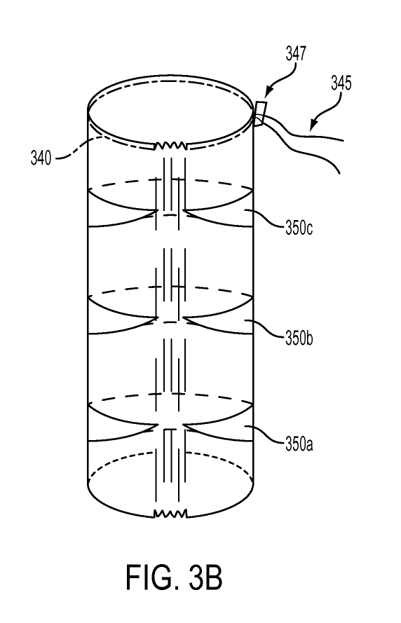

As illustrated in FIGS. 3A and 3B, each compression element 350a, 350b, 350c may extend circumferentially around the container to apply a radial compressive force on the container and its contents. The size, shape, and density of the distribution of the compression elements 350a, 350b, 350c along a length of the container 320 may be varied dependent upon the amount of compressive force to be applied to the container 310 and its contents. Thus, although illustrated in the present exemplary embodiment as including three compression elements, the container 310 may include fewer or more compressive elements. The compressive elements may vary in size, shape, and position (e.g., aligned or not aligned, spaced the same distance from one another or irregularly spaced, etc.) in order to provide a desired compressive force to the container and its contents. As will be appreciated by those of ordinary skill in the art, shape memory alloys can be used to allow the compression elements to move automatically between the first and second configurations.

Container 310 may include a closure system 340 for closing open end 320 (and, if open, end 330) of container 310 after a specimen is placed within the container. The closure system 340 may include any conventional structure for closing an open container. As illustrated in the exemplary embodiment of FIGS. 3A and 3B, closure system 340 may include a drawstring type closure element 345. Additionally, closure system 340 may include a clip or fastener type element 347 to maintain the closure element 345 in a closed configuration. As illustrated in FIG. 3B, the closure system 340 may reduce a size of the opening of open end 320. Additionally or alternatively, closure system 340 may completely close the opening of open end 320.

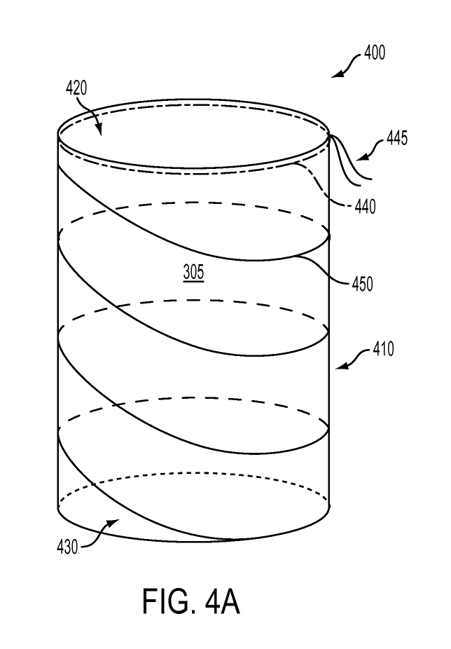

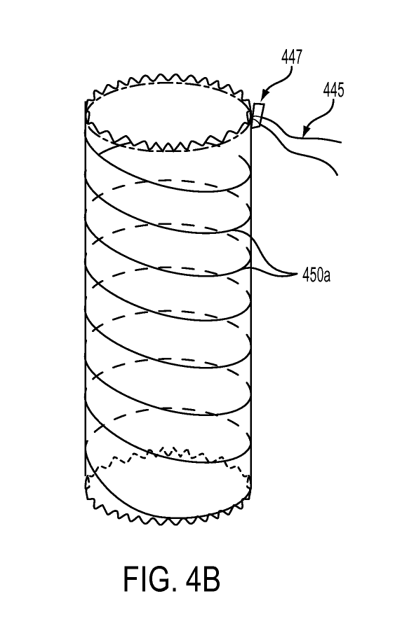

Additionally or alternatively, as illustrated in FIGS. 4A and 4B, in a specimen retrieval system according the present teachings, a compression structure may comprise a single shape memory alloy compression element 450a. As shown in the exemplary embodiment of FIG. 4A, a container 410 may include a single compression element 450a wound around container 410 in a spiral configuration. The density of the coil distribution along a length of the container 410 can vary (coil pitch; see, e.g., FIG. 4B), as can the thickness of the coil and a cross-sectional shape of the coil. The compression structure 450a may be movable between a first non-engaged configuration, in which a specimen receiving container 410 is open to receive a tissue specimen, and a second engaged configuration, in which the compressive element 450a is engaged to apply a compressive force to container 410, resulting in a reduced or altered dimension (e.g., diameter), size (e.g., cross-sectional area), shape or volume of the container 410, the shape alloy compression element 450a applying compression along a substantial length of the container 410, and via the container, compressing any tissue contained therein.

As with previous embodiments, container 410 may include a closure system 4340 for closing open end 420 (and, if open, end 430) of container 410 after a specimen is placed within the container. The closure system 440 may include any conventional structure for closing an open container. As illustrated in the exemplary embodiments of FIGS. 4A and 4B, closure system 440 may include a drawstring type closure element 445. Additionally, closure system 440 may include a clip or fastener type element 447 to maintain the closure element 445 in a closed configuration. As illustrated in FIG. 4B, the closure system 440 may reduce a size of the opening of open end 420. Additionally or alternatively, closure system 440 may completely close the opening of open end 420.

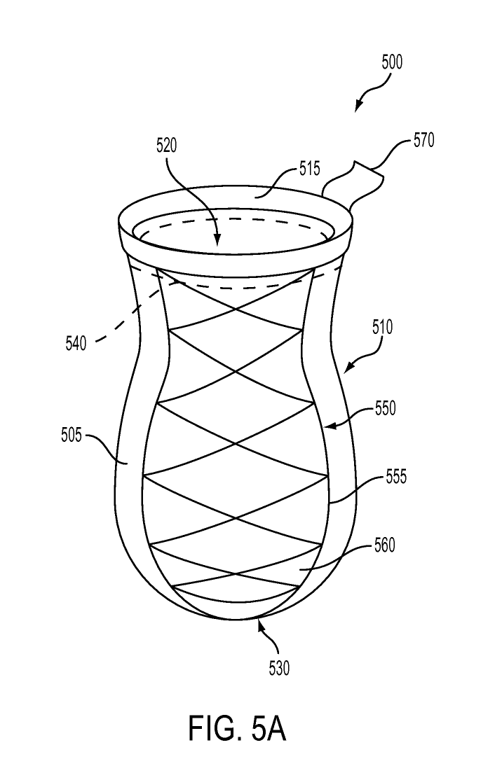

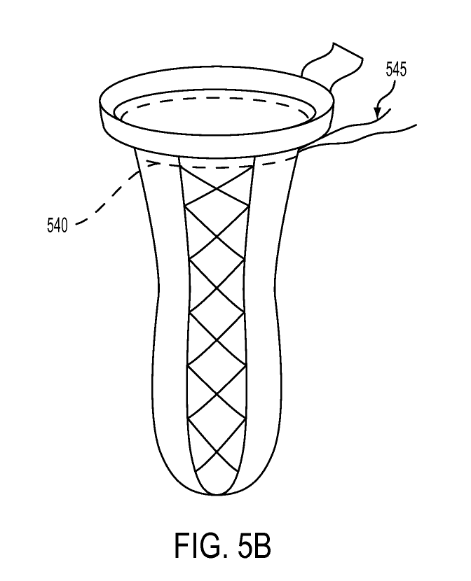

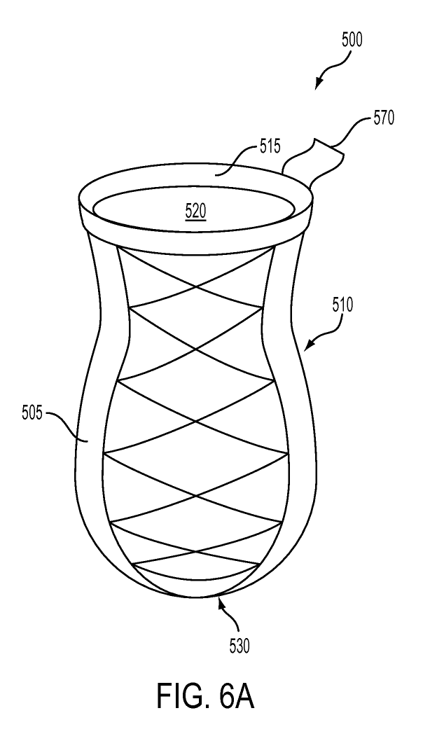

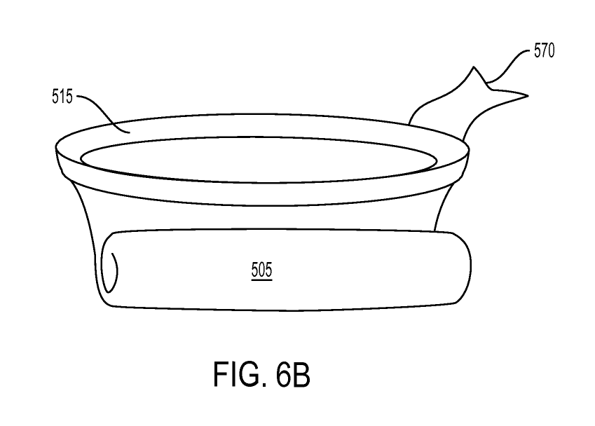

According to another aspect of the present teachings, a specimen retrieval system 500 includes a bag 510 having an open end 520 and a closed end 530. As shown in FIGS. 5A and 5B, open end 520 may include a semi-rigid, compressible ring-like structure 515 intended to maintain open end 520 in an open configuration. Ring 515 may be made from any suitable material, such as a rubber material. As discussed above, a body 505 of bag 510 may comprise any suitable material capable of containing excised surgical tissue. During delivery to the surgical site, body 105 of bag 510 may be rolled upon itself to present a profile that will fit through a surgical access site, such as through a trocar inserted in an incision. To permit passage through a trocar (not shown), ring 515 can be compressed, flattened, or otherwise manipulated. (See FIGS. 6A and 6B.) After passage through the trocar and into the surgical space, ring 515 will automatically resume its open position, holding end 120 open to receive the excised tissue. An additional closure element, such as closure element 540, may be provided to close the open end 120 of bag 510 adjacent to ring 515 after the excised tissue has been placed in bag 510. To assist with moving bag 510 within the surgical space, and to assist in withdrawing bag 510 from the surgical space, bag 510 can be provided with a tab element 570. Tab 570 provides a surface for surgical tools to grasp when manipulating bag 510 relative to the excised tissue, and relative to surgical access ports.

An exemplary method of using the specimen retrieval system in a minimally invasive hysterectomy surgery will now be described. In a minimally invasive surgical procedure, such as a manual or robotic endoscopy, surgical access ports are established. For a hysterectomy procedure, a first site, usually the endoscope access port, is established in a central location in the abdomen, often through the navel (umbilicus). Instrument access ports are established to the right and left of the endoscope access port. A uterine manipulator (see retractor/uterine manipulator identified in FIGS. 9-12) is positioned through the vaginal canal and positioned within the uterus to control movement of and to manipulate the uterus during the surgical procedure. The surgeon (and assistant(s)) utilizes instruments, either directly or indirectly, to free the uterus from connections to other tissues within the body. The ovaries and fallopian tubes may be left connected to the uterus for removal with the uterus, or may be separated from the uterus. After separation of the uterus from adjacent tissues and organs, the uterus is ready for removal. If the excised tissue is larger than the existing surgical access ports, including the vaginal canal, a specimen retrieval system is selected.

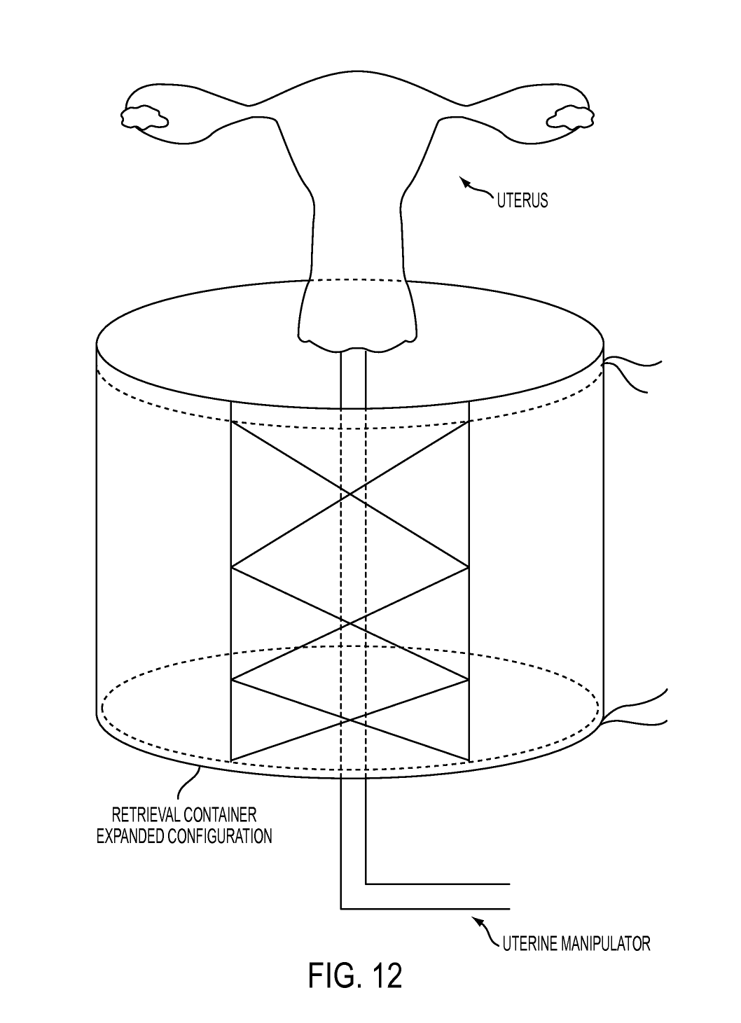

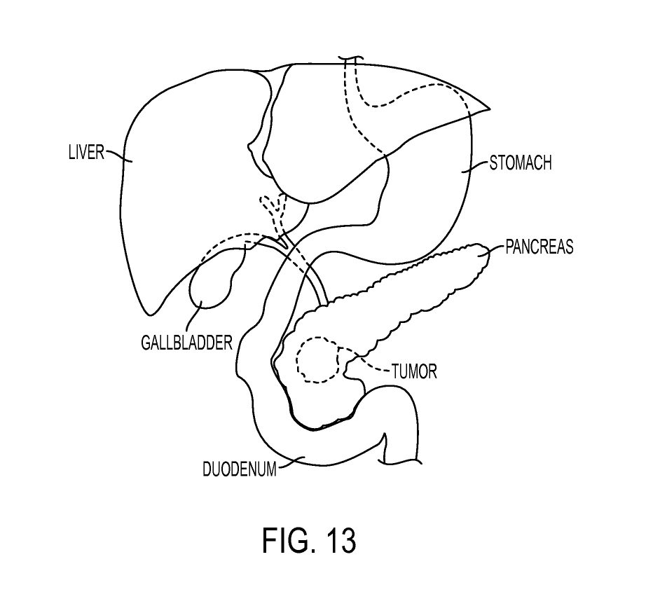

According to one aspect of the present teachings, a specimen retrieval system, such as that illustrated in FIGS. 1A and 1B, includes specimen retrieval sleeve 110 having a first open end 120 and a second open end 130. The sleeve 110 is advanced over the uterine manipulator, through the vaginal canal, and into the surgical space. (See FIG. 12.) The sleeve, in a first open configuration, is manipulated by the surgeon until it slides over and substantially encompasses the excised tissue to be removed from the surgical site. After positioning the sleeve 110 relative to the uterus (excised tissue), closure elements 140, located at the first and second ends 120, 130 of the sleeve 110 may be closed, for example by pulling on drawstring closure elements. When closed, the ends may be secured in the closed position by use of a clip 147. In certain cases, where shedding of cancerous cells are concern, it may be desirable to close ends 120, 130 of sleeve 110 to substantially completely contain the tissue being removed. In cases where contamination is not a concern, surgeons may choose to forego closing the ends of the sheath 110, choosing to focus on reducing a dimension such as a diameter of the sheath, and thus reducing a diameter of the tissue mass contained within the sheath 110, to facilitate removal through the vaginal canal.

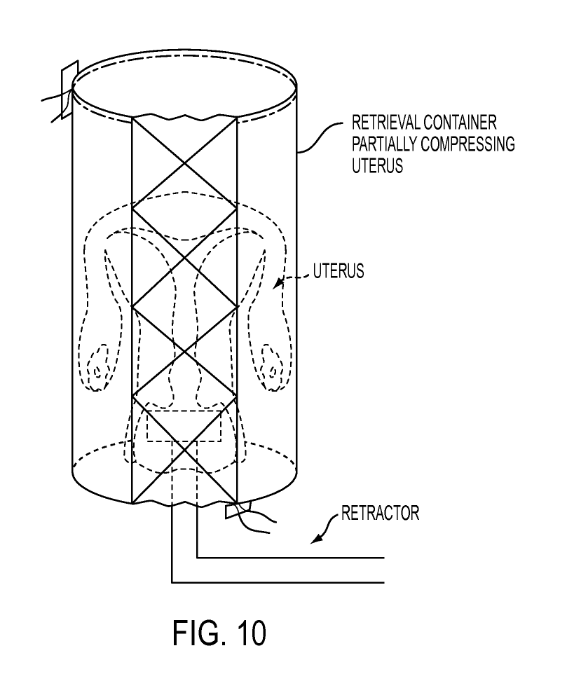

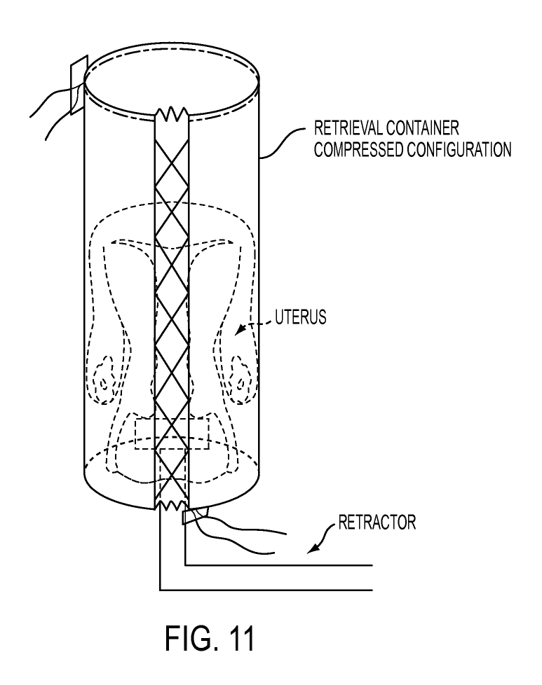

Next, the surgeon actuates the compression structure 150, grasping and pulling the drawstring 160 to draw the edges 155 of the lacing construction close to one another, thereby reducing or altering a dimension (e.g., diameter), a size (e.g., cross-sectional area), a shape, or a volume of the sleeve and applying a compressive force, via the compression structure, to the excised tissue contained in the sleeve 110. See FIGS. 10 and 11. After the tissue has been compressed within sleeve 110, sleeve 110 and the compressed tissue are withdrawn from the body, via the vaginal canal, using the uterine manipulator.

In one example, a sheath according to the present teachings may have first and second open ends, each open end having a diameter of about four (4) inches. The sheath may have a length of about seven (7) inches and a volume of about 250 cc. In order to allow removal of a sheath containing excised tissue through the vaginal canal, the sheath includes a compression structure configured to apply a compressive force along a length of the sleeve to reduce a diameter of the sleeve, with tissue contained therein, to less than three (3) inches, and to as little as two (2) inches. Such a reduction in diameter reduces the volume of the sheath and changes the shape of the excised tissue, compressing it into a substantially cylindrical shape. The amount of compression obtained when the compression structure is engaged will depend, in part, on the type of tissue (e.g., muscle or bone will be less compressible than a hollow organ). Surgeons can select sheaths with appropriate types of compression structure(s) depending upon the type of tissue intended for removal.

According to another aspect of the disclosure, a specimen retrieval system such as the one illustrated in FIGS. 5A, 5B, 6A, and 6B may include a specimen bag 510 having an open end 520 and a closed end 530. After separation of the uterus from adjacent tissues and organs, the uterus is ready for removal. The body of bag 510 is rolled upon itself, similar to rolling a sleeping bag, and the ring 515 at the top of the bag is compressed into a substantially flat configuration. The rolled and compressed bag 510 is inserted into the body through a trocar. Access may be provided through an existing surgical incision, or a new minimally invasive incision may be created. After passing though the trocar and into the body, the ring 515 on bag 510 resumes its open configuration, opening end 520 of the bag to receive the excised tissue. The surgeon or assistant may facilitate expansion of the bag by unrolling the body of the bag using a forceps or other surgical instrument. The surgeon then grasps tab 570 on bag 510 to position the bag 510 relative to the excised tissue (uterus). The uterus is positioned within the bag 510, and a top of the bag may be closed. A closure element 540, located at the open end 520 of the bag 510 may be closed, for example by pulling on drawstring closure elements. When closed, the end may be secured in the closed position by use of a clip 547. Next, the surgeon actuates the compression structure 550, grasping and pulling the drawstring 560 to draw the edges 555 of the lacing construction close to one another, thereby reducing or altering a dimension (e.g., diameter), a size (e.g., cross-sectional area), a shape, or a volume of the sleeve and applying a compressive force, via the compression structure, to the excised tissue contained in the bag 510. After the tissue has been compressed within bag 510, bag 510 and the compressed tissue are withdrawn from the body, either via the vaginal canal, using the uterine manipulator (retractor), or via another existing surgical access port.

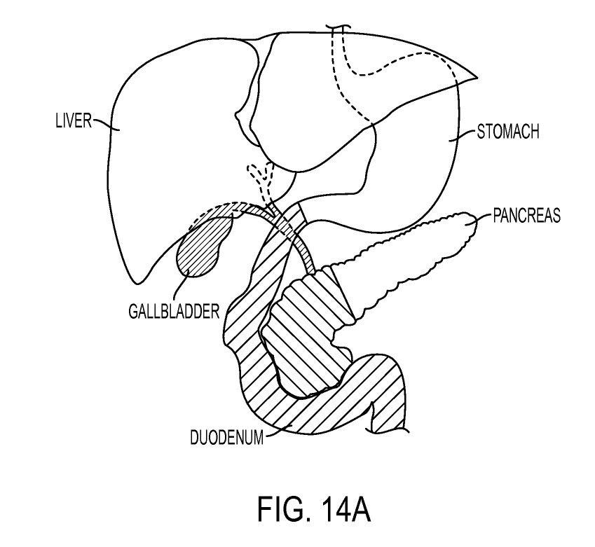

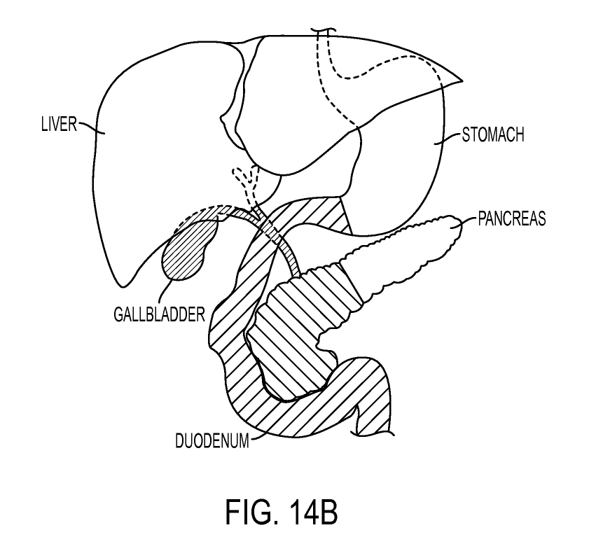

Another exemplary method of using the specimen retrieval system in a minimally invasive surgery will now be described. In a minimally invasive surgical procedure, such as a manual or computer-assisted endoscopy, surgical access ports are established. For a robotic or computer-assisted whipple procedure, five ports may be established in the patient's abdominal area: a first port for a camera, second and third ports surgical instrument access ports, and fourth and fifth assistant operating ports. After separation of the gall bladder, a portion of the pancreas (containing a tumor), a portion of the small intestine (duodenum), the bile duct, and in some cases, a portion of the stomach from adjacent tissues and organs, the excised tissue block is ready for removal. See FIGS. 14A and 14B, in which the cross-hatching indicates portions of the tissues and organs that will be removed during the procedure. If the excised tissue block has a dimension that is larger than the existing surgical access ports, such that it will be difficult to remove the excised tissue block through the existing access ports, a specimen retrieval system is selected.

According to one aspect of the present teachings, a specimen retrieval system, such as that illustrated in FIGS. 1A and 1B, includes specimen retrieval sleeve 110 having a first open end 120 and a second open end 130. The sleeve 110 is advanced into the surgical space through one of the access ports. The sleeve, in a first open configuration, is manipulated by the surgeon until it slides over and substantially encompasses the excised tissue block to be removed from the surgical site. After positioning the sleeve 110 relative to the excised tissue, closure elements 140, located at the first and second ends 120, 130 of the sleeve 110 may be closed, for example by pulling on drawstring closure elements. When closed, the ends may be secured in the closed position by use of a clip 147. In certain cases, where shedding of cancerous cells are concern, it may be desirable to close ends 120, 130 of sleeve 110 to substantially completely contain the tissue being removed. In cases where contamination is not a concern, surgeons may choose to forego closing the ends of the sheath 110, choosing to focus on reducing a dimension such as a diameter of the sheath, and thus reducing a diameter of the tissue mass contained within the sheath 110, to facilitate removal through an existing surgical access port.

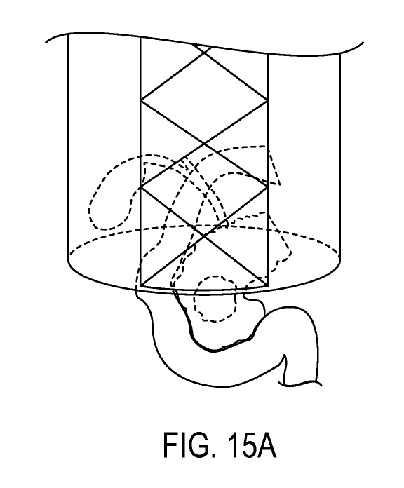

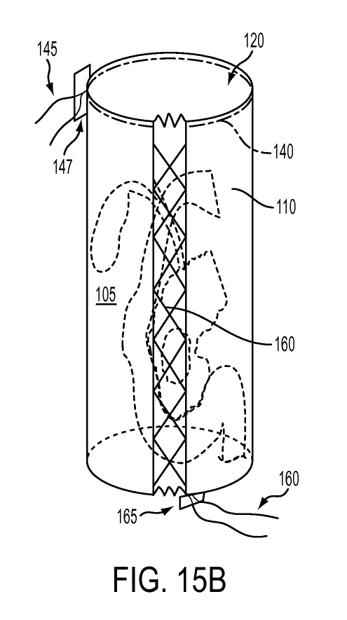

Next, the surgeon actuates the compression structure 150, grasping and pulling the drawstring 160 to draw the edges 155 of the lacing construction close to one another, thereby reducing or altering a dimension (e.g., diameter), a size (e.g., cross-sectional area), a shape, or a volume of the sleeve and applying a compressive force, via the compression structure, to the excised tissue contained in the sleeve 110. See FIGS. 15A and 15B. After the tissue has been compressed within sleeve 110, sleeve 110 and the compressed tissue are withdrawn from the body, via an existing surgical access port.

According to another aspect of the disclosure, a specimen retrieval system such as the one illustrated in FIGS. 5A, 5B, 6A, and 6B may include a specimen bag 510 having an open end 520 and a closed end 530. After separation of the gall bladder, a portion of the pancreas, a portion of the small intestine (duodenum), the bile duct, and in some cases, a portion of the stomach from adjacent tissues and organs, the excised tissue block is ready for removal. The body of bag 510 is rolled upon itself, similar to rolling a sleeping bag, and the ring 515 at the top of the bag is compressed into a substantially flat configuration. The rolled and compressed bag 510 is inserted into the body through a trocar. Access may be provided through an existing surgical incision. After passing though the trocar and into the body, the ring 515 on bag 510 resumes its open configuration, opening end 520 of the bag to receive the excised tissue. The surgeon or assistant may facilitate expansion of the bag by unrolling the body of the bag using a forceps or other surgical instrument. The surgeon then grasps tab 570 on bag 510 to position the bag 510 relative to the excised tissue. The excised tissue is positioned within the bag 510, and a top of the bag may be closed. A closure element 540, located at the open end 520 of the bag 510 may be closed, for example by pulling on drawstring closure elements. When closed, the end may be secured in the closed position by use of a clip 547. Next, the surgeon actuates the compression structure 550, grasping and pulling the drawstring 560 to draw the edges 555 of the lacing construction close to one another, thereby reducing or altering a dimension (e.g., diameter), a size (e.g., cross-sectional area), a shape, or a volume of the sleeve and applying a compressive force, via the compression structure, to the excised tissue contained in the bag 510. After the tissue has been compressed within bag 510, bag 510 and the compressed tissue are withdrawn from the body, via an existing surgical access port.

Further modifications and alternative embodiments will be apparent to those of ordinary skill in the art in view of the disclosure herein. For example, the systems and the methods may include additional components or steps that were omitted from the diagrams and description for clarity of operation. Moreover, those of ordinary skill in the art will appreciate that aspects and/or features disclosed with respect to one embodiment in some case may be incorporated in other embodiments even if not specifically described with respect to such other embodiments. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims. Accordingly, this description is to be construed as illustrative only and is for the purpose of enabling those skilled in the art the general manner of carrying out the present teachings.

It is to be understood that the particular examples and embodiments set forth herein are non-limiting, and modifications to structure, dimensions, materials, and methodologies may be made without departing from the scope of the present teachings. Other embodiments in accordance with the present disclosure will be apparent to those skilled in the art from consideration of the specification and practice of the invention disclosed herein. It is intended that the specification and examples be considered as exemplary only, with a true scope and spirit being indicated by the following claims.

What is claimed is:

1. A specimen retrieval system, comprising:
a specimen retrieval container comprising a first open end, a second end, and a body extending between the first end and the second end to define a length of the container; and
a compression structure extending along a substantial portion of the length of the container, the compression structure movable between a first configuration and a second configuration,
wherein, when in the second configuration, the compression structure applies a compressive force along a length of the container to reduce or alter at least one of a dimension, a size, a shape, and a volume of the container and thereby compress a tissue contained therein.

2. The specimen retrieval system of claim 1, wherein the retrieval container is a sleeve and the second end is an open end.

3. The specimen retrieval system of claim 1, wherein the compression structure is integrally formed with the specimen retrieval container.

4. The specimen retrieval system of claim 1, wherein the compression structure comprises a shape memory alloy.

5. The specimen retrieval system of claim 1, wherein the compression structure includes a plurality of reinforcing structures.

6. The specimen retrieval system of claim 5, wherein the compression structure further comprises a connecting element connecting the plurality of reinforcing structures.

7. The specimen retrieval system of claim 6, wherein the connecting element moves the plurality of reinforcing structures toward one another in the second configuration.

8. The specimen retrieval system of claim 6, wherein the connecting element reduces a diameter of the specimen retrieval container when in the second configuration.

9. The specimen retrieval system of claim 2, wherein the compression structure includes a lacing closure.

10. The specimen retrieval system of claim 1, wherein the compression structure is configured to apply the compressive force substantially evenly along the length of the container.

11. The specimen retrieval system of claim 1, wherein the compression structure is configured to substantially uniformly compress tissue contained in the container.

12. A method of removing excised tissue from an endoscopic surgical site, the method comprising:
inserting a specimen retrieval container into a patient;
positioning the specimen retrieval container relative to excised tissue at a surgical site within the patient;
substantially encompassing the excised tissue with the specimen retrieval container;
applying a force to the specimen retrieval container to reduce or alter at least one of a dimension, a size, a shape, and a volume of the specimen retrieval container and thereby compress the excised tissue contained therein.

13. The method of claim 12, wherein applying a force to the specimen retrieval container includes radially compressing the specimen retrieval container and the excised tissue contained therein.

14. The method of claim 12, wherein at least one of a dimension, a size, a shape, and a volume of the excised tissue is larger than an access to the endoscopic surgical site prior to applying the force.

15. The method of claim 14, wherein applying the force to the specimen retrieval container reduces or alters at least one of a dimension, a size, a shape, and a volume of the excised tissue to permit the excised tissue to exit the surgical site via the access.

16. The method of claim 12, wherein the access to the endoscopic surgical site is a vaginal canal.

17. The method of claim 16, further comprising removing the container and compressed tissue from the endoscopic surgical site via the vaginal canal.

18. The method of claim 12, wherein the excised tissue includes at least one of lung tissue, colon tissue, uterine tissue, gallbladder tissue, and appendix tissue.

19. The method of claim 12, wherein the specimen retrieval container is a sleeve, and wherein inserting a specimen retrieval container into a patient includes sliding the sleeve over a surgical instrument positioned in the patient and into the body.

20. The method of claim 18, wherein positioning the specimen retrieval container relative to the excised tissue includes sliding the sleeve from the surgical instrument onto the excised tissue.

21. The method of claim 12, further comprising allowing an end of the specimen retrieval container to automatically expand from a substantially closed configuration to an open configuration.

22. A specimen retrieval system, comprising:
a specimen retrieval bag comprising a first open end, a second closed end, and a body extending between the first end and the second end of the bag, the body of the bag having a delivery configuration, a specimen receiving configuration, and a specimen removal configuration; and
a compression structure for reducing or altering at least one of a dimension, a size, a shape, and a volume of the bag to change the configuration of the bag from the specimen receiving configuration to the specimen removal configuration.

23. The specimen retrieval system of claim 21, wherein the compression structure extends circumferentially around at least a portion of the bag.

24. The specimen retrieval system of claim 22, wherein the compression structure distributes a force along a length of the bag to reduce or alter at least one of a dimension, a size, a shape, and a volume of the bag and any excised tissue contained therein.

25. The specimen retrieval system of claim 23, wherein the open end of the bag is biased in an open position.

26. The specimen retrieval system of claim 21, wherein at least one of a dimension, a size, a shape, and a volume of the bag is smaller when in the delivery configuration than in the specimen receiving configuration and the specimen removal configuration.
